(19) United States
(12) Patent Application Publication
(10) Pub. No.: US 2010/0112096 A1
(43) Pub. Date: May 6, 2010

(54) DRY EXTRACTS OF PELARGONIUM SIDOIDES AND PELARGONIUM RENIFORME

(75) Inventors: Joachim Herrmann, Walldorf (DE); Marc Thöle, Karlsruhe (DE)

Correspondence Address: EDWARDS ANGELL PALMER & DODGE LLP, P.O. BOX 55874, BOSTON, MA, 02205, US

(73) Assignee: DR. WILLMAR SCHWABE GMBH & CO., KG, KARLSRUHE (DE)

(21) Appl. No.: 12/450,823

(22) PCT Filed: Apr. 4, 2008
(86) PCT No.: PCT/EP2008/002720

§ 371 Date: Oct. 13, 2009

(30) Foreign Application Priority Data
Apr. 17, 2007 (DE) 102007018079.0

(51) Int. Cl. A61K 36/185; A01H 5/00; A61P 1/00; A61P 11/00

(52) U.S. Cl. 424/725; 800/298

(57) ABSTRACT

The invention relates to production methods for obtaining dry extracts from Pelargonium sidoides and/or Pelargonium reniforme, extracts which may be obtained according to said method, and pharmaceutical products comprising such extracts.

The present invention relates to production methods for obtaining dry extracts from Pelargonium sidoides and/or Pelargonium reniforme, extracts obtained by said methods and preparations containing such extracts.

The preparations obtained from the pelargonium species Pelargonium sidoides and/or Pelargonium reniforme native to southern Africa are traditionally used in this region for the therapeutic treatment of respiratory disorders and gastrointestinal symptoms.

The efficacy of an aqueous-ethanolic liquid extract of the roots of Pelargonium sidoides, EPs 7630, in the treatment of infections of the respiratory tract and the ENT region has meanwhile been proven by numerous clinical studies and observations of practical application (Kolodziej et al., Deutsche Apotheker Zeitung 143 (12): 55-64 (2003)).

The effect of the extract is caused by several therapeutically active components. Tanning agents and coumarin derivatives are considered important therapeutic components in Pelargonium sidoides. Such components are also contained in extracts from Pelargonium reniforme.

Depending on their consistency, the European Pharmacopoeia classifies extracts into liquid (liquid extracts and tinctures), semi-solid (viscous extracts) and solid (dry extracts) preparations. Dry extracts are prepared by evaporation or removal of the solvent used for preparation and usually have a loss in drying or water content of 5 wt.-% maximum. They have many advantages vis-à-vis liquid and semi-solid extracts. They have better stability, are easier to handle and may be used for preparing solid galenic dosage forms. In particular, direct use of an aqueous-ethanolic liquid extract is ruled out in those cases where a liquid dosage form without alcohol is desirable, for example in the administration to children.

Dry plant extracts are, for example, known from EP 0 589 921 B 1 and EP 1 037 674. These dry extracts contain carrier substances, among other things.

EP 0 589 921 B 1 relates to thick and/or dry plant extracts having the same or a very similar active ingredient spectrum as a corresponding liquid extract, the use thereof and a method for producing the same. EP 0 589 921 B 1 is based on the problem that not all of the volatile drug ingredients of liquid extracts may be contained in the resulting thick and/or dry extracts due to evaporation of the solvent in case of conventional drying. In addition, the extracts disclosed may contain pharmaceutical excipients, carrier media and/or disintegrants. Preferred substances cited are, among others, mono- and/or polysaccharides and cellulose, cellulose derivatives, starch and starch derivatives. The addition of the excipients which takes place after removing the solvent of the original liquid extracts has the object of preventing the escape of volatile components to any significant extent during the subsequent processing to obtain pharmaceuticals.

EP 1 037 647 B 2 relates to dry medicinal plant extracts from Passiflora, Agnus castus, Crataegus, Gingko, stinging nettle extract, valerian, Cimicifuga root or rootstock and/or Cynara for peroral application wherein the non-volatile phase of the extract is bonded to a carrier I which is solid at room temperature and is selected from polyethylene glycols, polyvinyl alcohols, polyvidone acetate and/or polyvinyl pyrrolidone as well as a carrier II which is selected from alcohol-insoluble, water-insoluble, water-swellable carriers solid at room temperature and or alkaline earth metal and/or alkali metal carbonates including hydrogen carbonates in micro-disperse form and/or in the form of a semi-solid or solid solution, optionally in addition to other excipients and/or additives. Such extracts are characterised by a release of the plant ingredients which is defined with regard to extent and speed.

However, we are faced with a problem in the preparation of pelargonium dry extracts, namely that the dry extracts obtained by direct drying of pelargonium liquid extracts will not dissolve completely even in a large solvent excess in physiologically compatible, primarily aqueous and or aqueous-alcoholic solvents including mixtures of water and polyols and, optionally, alcohols (cf. comparative examples 1-2). On the one hand, this makes the production of liquid preparations from these dry extracts difficult, while the efficacy of the dry extracts may be generally affected on the other.

Therefore, it is the object of the present invention to provide dry extracts from Pelargonium sidoides and/or reniforme having improved solubility.

Dry extracts prepared by the method of the invention are at least somewhat soluble in physiologically compatible solvents. According to the European Pharmacopoeia, 5^{th} ed., they dissolve practically without residues at a ratio of at least 1 g of dry extract to 100 ml of solvent and thus yield a clear or opalescent solution without any sediment. Said opalescence is not higher than the opalescence reference suspension of the European Pharmacopoeia, 5^{th} ed. (corresponding to 60 NTU=Nephelometric Turbidity Units).

Surprisingly, it has now been found that the solubility of dry extracts from Pelargonium sidoides and/or Pelargonium reniforme is significantly improved if carrier substances selected from the group of saccharides and sugar alcohols are added to the extract solutions used before conversion to a solid form by drying. This effect is particularly surprising as the solution characteristics of dry extracts prepared by the conventional route in physiologically compatible solvents cannot be improved by simple admixing of these carrier substances (see comparative examples 3-8).

The improved solubility of the dry extracts of the invention is particularly advantageous if the dry extracts are processed with the customary excipients to obtain (coated) tablets. In this case, a particularly favourable release of the active ingredient can be achieved by using the dry extract of the invention. Typically, this will be demonstrated in accordance with the method 2.9.3.5 of the European Pharmacopoeia, 5^{th} ed., “Prüfung der Wirkstofffreisetzung aus festen Arzneiformen” (testing the release of active ingredients from solid dosage forms). A good release of the active ingredient from the dosage form is a prerequisite for a good efficacy.

The extract solutions of Pelargonium sidoides and/or Pelargonium reniforme (i.e. solutions of the starting extract) to be used in the method for preparing the dry extracts of the invention may be obtained, for example, by first extracting dried and comminuted roots of Pelargonium sidoides and/or Pelargonium reniforme with water and one or more aqueous-alcoholic solvents or one or more aqueous-ketonic (i.e. aqueous-acetonic) solvents by the conventional route, for example at temperatures of 10 to 100° C. Where necessary, the drug residue is slightly squeezed out and the crude extract optionally filtered. It is preferred to use mixtures of water and a monohydric C₁-C₃ alcohol selected from methanol, ethanol, 1-propanol and 2-propanol for preparing the solution of the starting extract.

The water portion of the aqueous-alcoholic or aqueous-ketonic solvents is preferably at least 50 wt.-% and preferably at most 95 wt.-%. It is preferred to prepare the liquid extract by percolation with an aqueous-ethanolic solvent, optionally after prior mashing with an aqueous-ethanolic solvent in accordance with EP 1 429 795.

Other suitable extract solutions are also described in DE 10 2004 063 910, for example, especially in para. [0017] and examples 3 and 4. The disclosure of the two latter publications is expressly included by reference with regard to the preparation of extract solutions.

After that, a solid carrier substance is dissolved in the liquid extract thus obtained. Alternatively, several solid carrier substances may be used. The mass ratio of the carrier substance(s) to the dry residue (determined in accordance with the European Pharmacopoeia, 5^{th} ed., by three hours of drying at 100 to 105° C.) of the extract solution is 1:4 to 9:1, preferably 1:1 to 6:1, especially 2:1 to 5:1. The solution is concentrated and dried by the usual methods, for example at a pressure of 0.001 bar to atmospheric pressure and a temperature of 20 to 100° C. Alternatively, the carrier substance(s) may be added during the concentration step.

Suitable carrier substances are monosaccharides such as fructose, galactose, glucose, xylose and/or oligosaccharides such as α-cyclodextrin, β-cyclodextrin, γ-cyclodextrin, hydroxypropyl betadex, lactose, lactulose, maltose, raffinose, saccharose, trehalose and/or polysaccharides such as chitosan, chitosan hydrochloride, dextran, dextrin guargalactomannan, gum arabic, hydroxyethyl cellulose, hydroxypropyl cellulose, hydroxypropylmethyl cellulose, inulin, maltodextrin, methylcellulose, methylhydroxyethyl cellulose, polydextrose and/or sugar alcohols such as erythritol, isomalt, lactilol, maltitol, mannitol, sorbitol, xylitol.

Another subject matter of the invention are dry extracts from Pelargonium sidoides and/or reniforme that may be obtained by the method of the invention.

Another subject matter of the invention are preparations containing said dry extracts, optionally in combination with other substances such as active ingredients and/or excipients.

These preparations may be drugs, food products, medical products, cosmetic products or consumer products, for example. Food products should especially be interpreted as dietetic food products, food supplements as well as medical food, health food and dietary supplements.

The dry extracts of the invention may be processed together with the customary excipients to obtain solid preparations such as powders, granulates, pellets, tablets, capsules or coated tablets. Excipients suitable for use may be the customary fillers, binders, disintegrants, lubricants and, optionally, aroma and flavouring agents and coating agents for coated tablets. The customary excipient oils and fats may be used as fillers in the preparation of soft capsules; the shell of the soft capsules may be made of gelatine, for example. The dry extracts according to the invention may be processed with the customary excipients to obtain liquid preparations such as solutions, sprays, emulsions and suspensions. Common solvents, solubilisers, stabilisers as well as aroma and flavouring agents may be used as excipients. Dosing is selected in such a manner that a quantity of the dry extract is taken per day which corresponds to 2 to 1,000 mg, preferably 5 to 400 mg, and especially preferably 10 to 200 mg of dry residue of the liquid extract used for preparation.

### EXAMPLES

The following solvents A and B were used in the comparative examples 1 to 8 and the examples 9 to 14:

### Solvent A:

| | |
|---|---|
| Ethanol 96 vol.-% | 10 parts by mass |
| Glycerol 85 wt.-% | 20 parts by mass |
| Water | 70 parts by mass |

### Solvent B:

| | |
|---|---|
| Glycerol 85 wt.-% | 10 parts by mass |
| Xylitol | 10 parts by mass |
| Water | 80 parts by mass |

### Comparative Examples 1 to 8

28 kg of ethanol (35 wt.-%) were added to 14 kg of ground root of Pelargonium sidoides and stored at room temperature for 20 hrs. Afterwards, the mixture was percolated with 112 kg of ethanol (6 wt.-%) for 10 hrs and then filtered. The dry residue of the filtrate was 1.78 wt.-%.

50 kg of this liquid extract were dried at 50° C. under vacuum (up to 18 mbar).

1 g each of the dry extracts obtained was mixed with 100 ml of the solvent A or B, optionally after thorough mixing with 4.55 g of a carrier substance in a mortar.

| | Comparative | | | | | | | |
|---|---|---|---|---|---|---|---|---|
| | example No. | | | | | | | |
| | 1 | 2 | 3 | 4 | 5 | 6 | 7 | 8 |
| Dry extract | 1.00 g | 1.00 g | 1.0 g | 1.00 g | 1.00 g | 1.00 g | 1.00 g | 1.00 g |
| Mannitol | — | — | 4.55 g | 4.55 g | — | — | — | — |
| Saccharose | — | — | — | — | 4.55 g | 4.55 g | — | — |
| Maltodextrin | — | — | — | — | — | — | 4.55 g | 4.55 g |
| Supernatant | 1.5 | 6.5 | 1.84 | 3.8 | 1.8 | 4.2 | 14 | 115 |
| opalescence | | | | | | | | |
| (NTU) | | | | | | | | |
| Solvent | A | B | A | B | A | B | A | B |
| Sediment | + | + | + | + | + | + | + | + |

The dry extract was not completely soluble. All of the solutions showed a sediment.

### Examples 9 to 10

### Examples According to the Invention

28 kg of ethanol (35 wt.-%) were added to 14 kg of ground root of Pelargonium sidoides and stored at room temperature for 20 hrs. Afterwards, the mixture was percolated with 112 kg of ethanol (6 wt.-%) for 10 hrs and then filtered. The dry residue of the filtrate was 1.78 wt.-%.

1.25 kg of mannitol were dissolved in 15.4 kg of this liquid extract. The solution was dried at 50° C. under vacuum (up to 18 mbar).

5.55 g each of the dry extracts obtained (corresponding to 1 g of the native portion and 4.55 of mannitol) were mixed with 100 ml of solvent A or B.

Example No.

| | 9 | 10 |
|---|---|---|
| Dry extract with mannitol | 5.55 g | 5.55 g |
| Opalescence of the solution | 3.2 | 2.6 |
| (NTU) | | |
| Solvent | A | B |
| Sediment | — | — |

The dry extract dissolved completely. Both solutions showed no sediment.

### Examples 11 to 12

### Examples According to the Invention

28 kg of ethanol (35 wt.-%) were added to 14 kg of ground root of Pelargonium sidoides and stored at room temperature for 20 hrs. Afterwards, the mixture was percolated with 112 kg of ethanol (6 wt.-%) for 10 hrs and then filtered. The dry residue of the filtrate was 1.78 wt.-%.

1.19 kg of saccharose were dissolved in 14.7 kg of this liquid extract. The solution was dried at 50° C. under vacuum (up to 18 mbar).

5.55 g each of the dry extracts obtained (corresponding to 1 g of the native portion and 4.55 of saccharose) were mixed with 100 ml of solvent A or B.

Example No.

| | 11 | 12 |
|---|---|---|
| Dry extract with saccharose | 5.55 g | 5.55 g |
| Opalescence of the solution | 4.2 | 2.0 |
| (NTU) | | |
| Solvent | A | B |
| Sediment | — | — |

The dry extract dissolved completely. Both solutions showed no sediment.

### Examples 13 to 14

### Examples According to the Invention

28 kg of ethanol (35 wt.-%) were added to 14 kg of ground root of Pelargonium sidoides and stored at room temperature for 20 hrs. Afterwards, the mixture was percolated with 112 kg of ethanol (6 wt.-%) for 10 hrs and then filtered. The dry residue of the filtrate was 1.78 wt.-%.

1.34 kg of maltodextrin were dissolved in 16.5 kg of this liquid extract. The solution was dried at 50° C. under vacuum (up to 18 mbar).

5.55 g each of the dry extracts obtained (corresponding to 1 g of the native portion and 4.55 of maltodextrin) were mixed with 100 ml of solvent A or B.

Example No.

| | 13 | 14 |
|---|---|---|
| Dry extract with maltodextrin | 5.55 g | 5.55 g |
| Opalescence of the solution | 4.7 | 33 |
| (NTU) | | |
| Solvent | A | B |
| Sediment | — | — |

The dry extract dissolved completely. Both solutions showed no sediment.

1. Method for preparing a dry extract from Pelargonium sidoides and/or Pelargonium reniforme with improved solubility, characterised by the following process steps:
(a) preparing an aqueous or aqueous-alcoholic or aqueous-ketonic solution of a starting extract from Pelargonium sidoides and/or Pelargonium reniforme, the alcohol in the aqueous-alcoholic solution being a monohydric C₁-C₃ alcohol selected from methanol, ethanol, 1-propanol and 2-propanol,
(b) adding a solid carrier substance or several solid carrier substances selected from the group of monosaccharides and/or oligosaccharides and/or polysaccharides and/or sugar alcohols, the mass ratio of the carrier substance to the dry residue of the solution of the starting extract being 1:4 to 9:1; and
(c) evaporating and drying the extract solution thus obtained to yield the dry extract.

2. Method according to claim 1 wherein water-methanol mixtures, water-ethanol mixtures, water-1-propanol mixtures, water-2-propanol mixtures or water-acetone mixtures are used to prepare the solution of the starting extract.

3. Method according to claim 1 wherein water-methanol mixtures, water-ethanol mixtures, water-1-propanol mixtures, water-2-propanol mixtures or water-acetone mixtures are used to prepare the solution of the starting extract, the water proportion of the mixtures being at least 50 wt.-%.

4. Method according to claim 2, wherein the water proportion of the mixture used for preparing the solution of the starting extract is 95 wt.-% maximum.

5. Method according to claim 1, wherein the mass ratio of the carrier substance to the dry residue of the solution of the starting extract is 1:1 to 6:1.

6. Method according to claim 1 wherein the mass ratio of the carrier substance to the dry residue of the solution of the starting extract is 2:1 to 5:1.

7. Method according to claim 1 wherein the carrier substance is or the carrier substances are independently selected from the group comprising fructose, galactose, glucose, xylose, α-cyclodextrin, β-cyclodextrin, γ-cyclodextrin, hydroxypropyl betadex, lactose, lactulose, maltose, raffinose, saccharose, trehalose, chitosan, chitosan hydrochloride, dextran, dextrin guargalactomannan, gum arabic, hydroxyethyl cellulose, hydroxypropyl cellulose, hydroxypropylmethyl cellulose, inulin, maltodextrin, methylcellulose, methylhydroxyethyl cellulose, polydextrose, erythritol, isomalt, lactilol, maltitol, mannitol, sorbitol, and xylitol.

8. Dry extract from Pelargonium sidoides and/or Pelargonium reniforme which may be obtained according to claim 1.

9. Preparation containing a dry extract according to claim 8 and additional components allowed for the respective use.

10. Pharmaceutical product containing a dry extract according to claim 8 and other components allowed for pharmaceuticals.

11. Food product containing a dry extract according to claim 8 and other components allowed for food products.

12. Medical product containing a dry extract according to claim 8 and other components allowed for medical products.

13. Cosmetic product containing a dry extract according to claim 8 and other components allowed for cosmetic products.

14. Consumer product containing a dry extract according to claim 8 and other components allowed for consumer products.
